(19) United States
(12) Patent Application Publication
(10) Pub. No.: US 2012/0103342 A1
(43) Pub. Date: May 3, 2012

(54) Multidirectional tracheotomy speaking valve

(75) Inventors: Alan H. Shikani, Ruxton, MD (US); Frederick L. De Baugh, Forest Hill, MD (US)

(73) Assignee: SHIKANI MEDICAL, LLC d/b/a/ The Airway Company

(21) Appl. No.: 12/928,648

(22) Filed: Dec. 16, 2010

Related U.S. Application Data
(60) Provisional application No. 61/456,032, filed on Nov. 1, 2010.

(51) Int. Cl. A61M 16/20

(52) U.S. Cl. 128/207.16

(57) ABSTRACT

A speaking tube for a patient's airway management having a body with a chamber formed therein connected to a tracheotomy tube. A plurality of ramps are disposed within the chamber retaining a ball therein. The body is rotatable through 180° to adjust the positioning of the ball when the patient inhales and exhales, thereby facilitating speech by the patient. An adapter for heat moisture exchange is attachable to the body.

### CROSS REFERENCE TO RELATED APPLICATIONS

The present application is related to provisional patent application Ser. No. 61/456,032 filed Nov. 1, 2010, the disclosure and contents of which are included by reference herein in their entirety.

### BACKGROUND OF THE INVENTION

1. Field of the Invention

The present invention is directed to a speaking valve that is attached to a tracheotomy tube and more specifically to a valve which permits the patient to select a mode of use for ease of breathing and for speech. Also, the valve can be coupled with a heat moisture exchange filter.

Tracheotomy is a surgical procedure which is frequently performed to relieve obstruction of airflow through the larynx and upper trachea. One of its main side effects is loss of essential breathing functions including warming and filtering of air, coughing, smelling, tasting, swallowing, and more devastatingly, speaking. Voice production requires vibration of the vocal cords from a stream of air passing through the larynx. When a tracheotomy is present, exhaled air follows the path of least resistance, and goes through the tube, limiting the vibratory movement of the vocal cords, and hence limiting perceptual speech. This creates a psychological hardship, as communication is critical to patients' overall medical care and social interactions. This problem can be particularly disruptive in children, where tracheotomy can actually impact the development of normal language skills.

In order to redirect the air through the vocal cords, the patient may use a finger to occlude the tracheotomy tube. Finger occlusion however has several limitations: it requires manual dexterity (which some patients may lack); it also requires coordination of phonation with breathing (which some patients may be unable to perform); and it is unsanitary. The use of a tracheotomy speaking valve enables tracheotomy patients to speak without having to occlude the tracheotomy tube with their finger. Unidirectional speaking valves have a displaceable element that allows air to flow through the cannula and into the lungs during inspiration and prevent air from flowing through the cannula during expiration. Thus, during expiration, air flows through the patient's upper airways, such as the sub-glottic trachea, larynx, pharynx, mouth and nasal passages. As a result, tracheotomized individuals using a unidirectional tracheotomy valve are able to communicate orally and maintain clear upper airway passages by coughing or expelling air through the upper airway passages.

2. Description of Related Art

Prior U.S. Pat. No. 5,505,198 (Siebens et al) describes a unidirectional tracheotomy speaking valve with an external cylindrical housing chamber that contains a ball acting as the displaceable element. The ball moves back and forth during inspiration and expiration, and is limited from going beyond the housing chamber during inspiration by a pin or a wire that extends into the chamber and intersects a path of travel of the ball, preventing it from entering the patient's airway. In this patent, the housing chamber is external to the tracheotomy tube and attached to the cannula of a tracheotomy tube through coupling. U.S. Pat. No. 6,588,428 (Shikani et al) describes a similar design unidirectional speaking valve in which the housing chamber is internal and an integral part of the inner cannula of the tracheotomy tube, the ball is guided by longitudinal ribs and is restrained in the cannula by a wire.

While these patents constitute a substantial improvement in the art, nevertheless, there are certain inherent problems, as follows: 1) The noisiness associated with the ball regularly hitting on a metal pin or wire, which could be quite disruptive; 2) The risk of the pin or wire breaking with subsequent danger of ball aspiration in the patient's airway; 3) The fact that the valve housing chamber is a simple cylindrical tube that houses a ball that travels back and forth along the tube's central axis, with no guiding ribs that could potentially direct the movement of the ball depending on the orientation of the valve (valve “up” or “down”) on the position of the patient's neck, or on the needs of the patient vis-à-vis breathing and/or speaking.

In tracheotomy patients, because inhaled air enters the trachea directly, it is very important for health reasons and for the patient's comfort, that the inhaled air is at substantially the same temperature and contains the same quantities of moisture and dust as if it had reached the trachea after passing through the upper airway (nostrils, nose, pharynx and larynx), meaning a temperature approaching 32° Celsius with a moisture content approaching saturation at the temperature of this air and substantially free of dust. HME's (Heat Moisture Exchanger) enable this result to be achieved to some degree; the filter mass blocks a major part of the dust in suspension in the air, of course,; the water vapor contained in the patient's exhaled air, which is saturated at the temperature of the organism, condenses on the filter mass which is therefore heated substantially to the body temperature; inhaled air, arriving at the temperature of the ambient air, is warmed and takes up moisture in contact with the filter mass which is at a higher temperature and contains the condensed water.

In order to overcome these undesirable side effects of a tracheotomy procedure, the passive HME was developed and has been available for many years. An HME consists of a housing to direct exhaled airflow from the patient, through one of many types of humidifying and moisturizing media. This device is placed externally in between the outside air and the patient's air intake at the tracheotomy tube. Exhaled air from the patient enters the HME, is directed across the media. The media serves to absorb and retain moisture from the exhaled air. On inspiration, humidified and warmed air is then breathed in by the patient, thus achieving some of the effect of the natural nasal passage. The ebb and flow of air across its surface allows a recurring transference of moisture from the patient's exhaled air to the HME and back to the patient. However, the HME is not useable with a diaphragm-type valve which is unidirectional because these valves are always closed on expiration (bias closed) and do not allow exhaled air to flow through the valve on expiration.

### BRIEF SUMMARY OF THE INVENTION The present invention provides an improved unidirectional tracheotomy valve with substantial advantages heretofore not available in the prior art, as follows:

1. The pin/wire limiting mechanism is replaced with eccentrically positioned ramps that act as a stop mechanism but also act as a dynamic guide that directs the ball towards the front or the back of the chamber, depending on the position of the valve (valve “up” or valve “down”) correlating with the respective physiologic needs of the specific patient. This reduces the force required to move the ball with inhalation or exhalation, a particularly valuable feature in children and in patients having relatively low tidal volumes or limited pulmonary capacity.

2. The eccentric ramps allow a method of using the speaking valve in two different positions, and providing a positive ball positioning feature depending how the housing chamber is rotated, hence greatly improving performance. In one mode, with the valve oriented “down”, the ball is automatically held fully seated towards the front opening of the valve body, when the patient is breathing regularly at rest. This innovation allows the ball to sit inside the frontal opening and provide a leak free seal to the valve with no expiratory air required to seat the ball in the opening (“biased-closed position”). In the other mode with the valve oriented “up”, the ball has a tendency to sit away from the frontal opening, closer to the posterior opening of the chamber, providing a more open airflow passage (“biased-open position”) hence allowing the patient to breathe easier.

Additionally, the ball now requires a conscious effort in terms of exhalation force, to seat the ball in the frontal hole and seal off airflow. Because of this, exhaled air can either be allowed to exit through the valve rather than being redirected through the patient's upper airway. Alternately, the patient can force the ball to seat when re-direction of airflow is desired for speech production.

In the “bias open” position, exhaled air flows freely through the valve, hence allowing the improved tracheotomy speaking valve to be coupled/effective with a new Heat Moisture Exchange (HME) filter that fits over the improved speaking valve as a cap, enabling the patient to breathe inhaled air that is at substantially the same temperature and containing the same quantities of moisture and dust as if it had reached the trachea after passing through the upper airway (nostrils, nose, pharynx and larynx).

It is an object of the present invention to provide a speaking valve for a tracheotomy tube which has, in the “bias open” position reduced air resistance as compared to the prior art.

It is a further object of the present invention to provide a choice for the patient to select a mode for speech (“bias closed” position).

It is still a further object of the present invention to provide a speaking valve which can be coupled to a Heat Moisture Exchange (HME) filter.

In accordance with the teachings of the present invention there is disclosed a speaking valve for management of a patient's airway comprising a body removably attached to a tracheotomy tube. A plurality of circumferentially-spaced ramps are disposed within the body forming a chamber therein. The body has a first end communicating with the tracheotomy tube and a second end distal from the tracheotomy tube. An opening is formed in the second end offset from a central axis of the chamber. A ball having a diameter is disposed within the chamber eccentrically of the central axis of the chamber, the ball being guided up and down the ramps and substantially closing the opening in the second end of the body. Means are provided for rotating the body and hence the chamber therein, at the option and control of the patient wherein the ball may move up and down the ramps, opening and closing the opening in the second end of the body. A selected exhalation by the patient proceeds through the patient's upper respiratory system and facilitates speaking by the patient.

In further accordance with the teachings of the present invention, there is disclosed in a speaking valve for a tracheotomy tube for a patient, the combination of a body having a chamber formed about a central longitudinal axis and having a frontal opening. A ball is eccentrically disposed within the chamber and, in the biased-open position the ball is seated posteriorly inside the chamber, away from the frontal opening, hence resulting in less interference with airflow and easier inhalation. Means are provided for rotating the body approximately 180° about the central longitudinal axis thereof, and at the patient's or caregiver's option, to thereby automatically direct the ball forward against the frontal opening to thereby seal the frontal opening in a biased-closed position. In this manner, the inhalation is through the patient's upper respiratory system and over the patient's vocal cords, thereby facilitating substantially-improved speech by the patient.

In still further accordance with the teachings of the present invention, there is disclosed in a tracheotomy tube for a patient, the combination of a speaking valve operable in a biased-open or biased-closed position, respectively, at the option of the patient or the patient's caregiver, and a heat moisture exchange (HME) device cooperating with the speaking valve to facilitate improved airway management for the patient consonant with substantially-improved speech.

There is further disclosed, in accordance with the teachings of the present invention, a speaking valve for a patient's airway management, comprising a body having a chamber with a ball disposed therein eccentrically of the central axis of the chamber. Means including a plurality of circumferentially-spaced ramps for guiding the ball up the ramps upon initial inhalation by the patient are provided. Means are provided for rotating the body and hence the chamber therein, at the option of the patient. During the exhalation process, the patient's exhaled air causes the ball to ride up the frontal wall, seating in and sealing the opening. This redirects the exhaled air to exit through the laryngeal opening, thus enabling speech.

In addition, in accordance with the teachings of the present invention, there is disclosed a speaking valve for use with a tracheotomy device. A body has a proximate end and a distal end, respectively. The distal end of the cylinder has an opening formed therein. The body has a ramp means formed therein. A ball is disposed in the body between the ramp means and the opening and trapped therein. The ramp means includes a pair of substantially-parallel ramps spaced laterally apart a distance which is less than the diameter of the ball.

These and other objects of the present invention will become apparent from a reading of the following specification taken in conjunction with the enclosed drawings.

### BRIEF DESCRIPTION OF THE DRAWINGS

FIG. 1 is a perspective view showing a tracheotomy tube worn by a patient.

FIG. 2 is a perspective view of a tracheotomy tube with the valve of the present invention.

FIG. 3 is a cross-section taken along the lines 3-3 of FIG. 2 with the indexing means in a down position.

FIG. 4 is a cross-section view taken along the lines 4-4 of FIG. 3.

FIG. 5 is a partial cross-section view of the tracheotomy to be worn by the patient with the indexing means of the present invention in the down position and showing airflow when the patient stops inhaling.

FIG. 6 is a cross-section view corresponding to the view taken across the lines 3-3 of FIG. 2.

FIG. 7 is a cross-section view across the lines 7-7 of FIG. 6.

FIG. 8 is a partial cross-section view of the tracheotomy tube worn by the patient with the indexing means of the present invention in the down position showing airflow when the patient inhales.

FIG. 9 is a cross-section view corresponding to the view taken across the lines 3-3 of FIG. 2, however, with the indexing means in the up position when the patient inhales.

FIG. 10 is a cross-section view across the lines 10-10 of FIG. 9.

FIG. 11 is a partial cross-section view of the tracheotomy tube worn by the patient with the indexing means of the present invention in the up position showing airflow when the patient inhales.

FIG. 12 is a cross-section view corresponding to the view taken across the lines 3-3 of FIG. 2, however, showing the indexing means in the up position when the patient exhales.

FIG. 13 is a partial cross-section view of the tracheotomy tube worn by the patient with the indexing means of the present invention in the up position showing airflow when the patient exhales.

FIG. 14 is a cross-section view corresponding to the view taken across the lines 3-3 of FIG. 2, however, the indexing means is in the up position when the patient exhales with increased force.

FIG. 15 is a partial cross-section view of the tracheotomy tube worn by the patient with the indexing means of the present invention in the up position showing airflow when the patient exhales with increased force.

FIG. 16 is a cross-section view showing tab on the ring received in notch in the body.

FIG. 17 is an exploded view of the valve of the present invention.

FIG. 18 is a perspective view showing a protrusion as the indexing means.

FIG. 19 is a perspective view showing a Heat Moisture Exchange attachment to the valve.

### DETAILED DESCRIPTION OF THE INVENTION

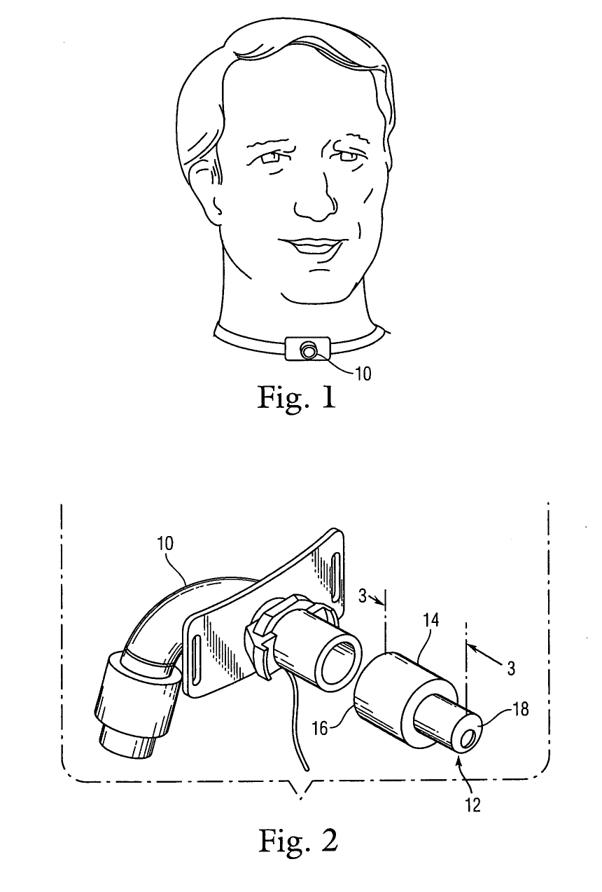

Referring to FIG. 1, a tracheotomy tube 10 is surgically implanted in the throat of a patient for airway management. On the end of the tracheotomy tube extending outwardly from the patient's throat at an angle of approximately 20°, there is removably-mounted the speech valve 12 of the present invention (FIG. 2).

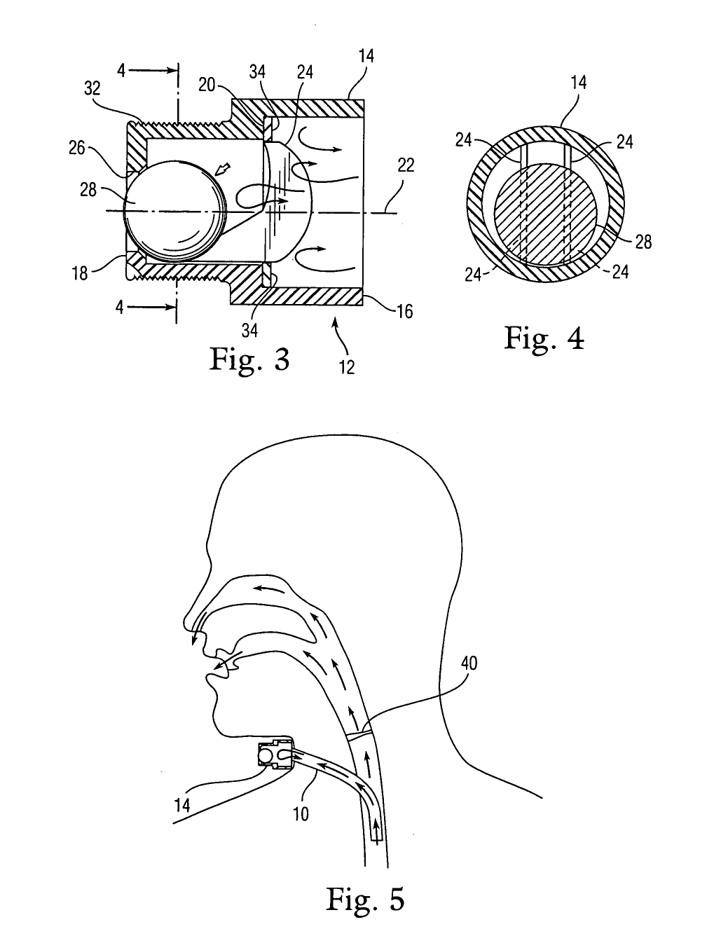

The speech valve 12 has a body 14 with a first end 16 which communicates with the outer end of the tracheotomy tube 10. The diameter of the first end 16 of the body is larger than the diameter of the second end 18 of the body forming a chamber having an internal step 20 within the body 14 (as shown in FIG. 3). The first end of the body is open. The second end 18 of the body 14 has a frontal opening 26 formed therein which is offset from the central axis 22 of the body. Within the body are a plurality of circumferentially spaced ramps 24. A portion of each ramp slopes upwardly at an acute angle toward the first end of the body. Preferably, there are two parallel ramps 24, although more or fewer ramps may be used. Within the body, between the frontal opening 26 and the ramps 24, there is disposed a ball 28. The ball has a diameter which is larger than the diameter of the frontal opening 26 in the second end of the body. The ramps are spaced apart a distance which is less than the diameter of the ball 28 to retain the ball within the body 14. The ramps form a channel or guide to keep the ball along a midline axis 22, making movement of the ball less turbulent and more efficient. The ramps have a defined slope to hold the ball fully forward toward the frontal opening 26 as will be described. The direction of movement of the ball is shown by the open arrow. Thus, in an “in rest” position when the body 14 is in a desired approximately horizontal position, the ball 28 is automatically seated against the frontal opening 26, thereby sealing the frontal opening when the patient is neither inhaling nor exhaling (FIGS. 3-5). The air passageway in the patient's upper airway is open for the passage of air and air passes over the vocal cords 40 enabling the patient to speak.

There is an indexing means 30 formed on the second end 18 of the body which is used to determine the orientation of the body 14. The body may be rotated through 180° by the patient (or the patient's caregiver) to provide an “up” and a “down” position of the body. The outer surface of the body may have threads 32 or ribs formed thereon to provide a better grip to rotate the body.

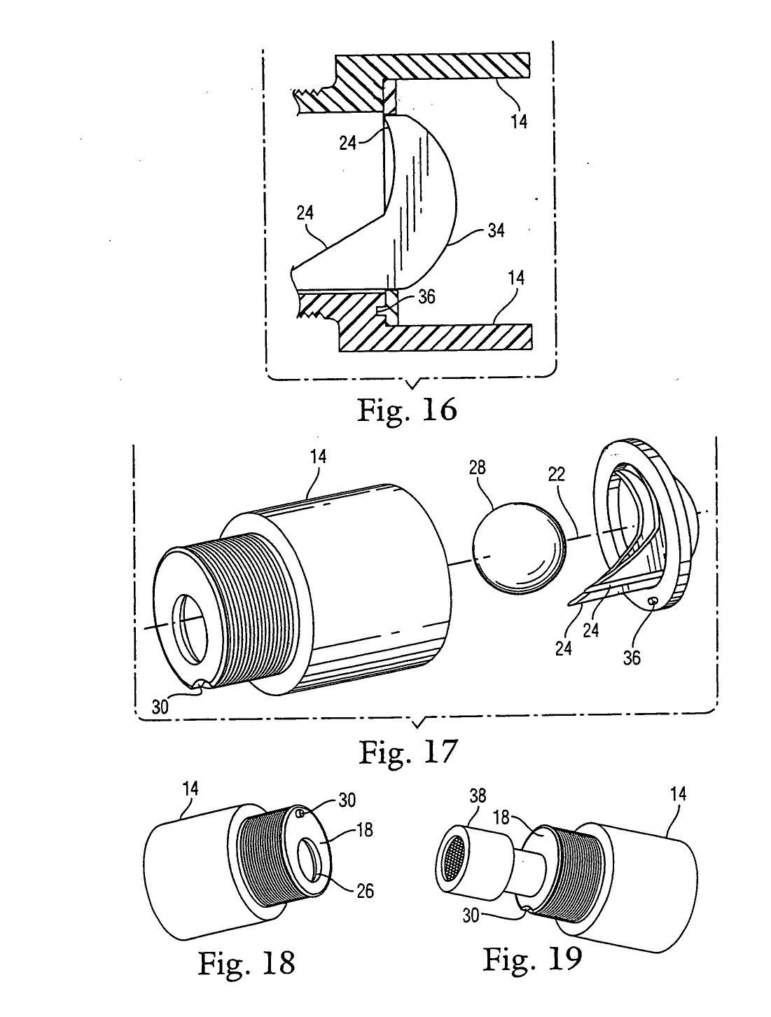

In a preferred embodiment the indexing means 30 is a notch or non-round portion of the second end of the body located near the frontal opening in the body. Alternately, as shown in FIG. 16, the indexing means may be a protrusion extending outwards from the second end of the body. Other indexing means known to persons skilled in the art may be used to provide an indexing means that may be sensed tactilely by the patient.

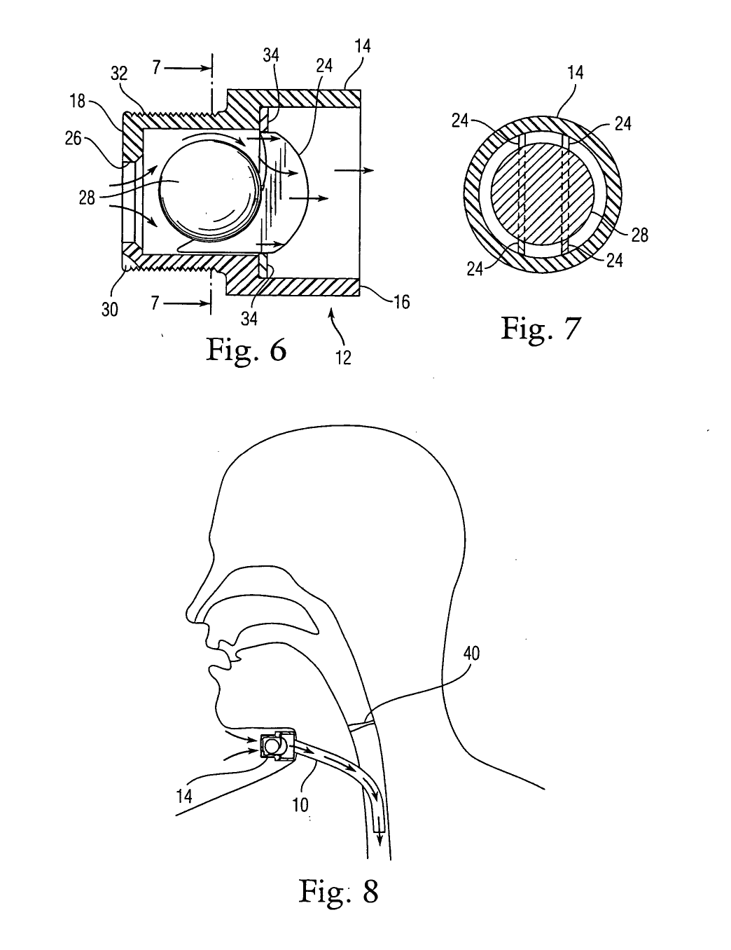

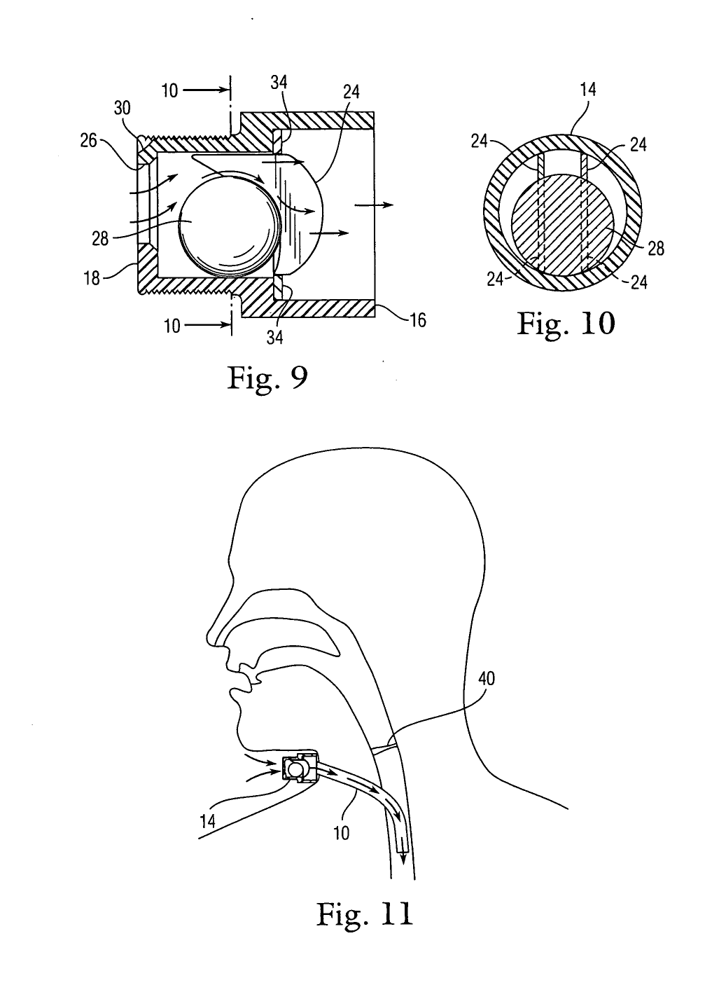

As shown in FIGS. 6-8, when the indexing means in the “down” position, and the patient inhales, the incoming air moves the ball up the ramps 24 toward the tracheotomy tube 10 and air flows around the ball, between the ramps and into the patient's lungs. With the indexing means in the “up”/biased-open position (FIGS. 9-11), and the patient in the resting position, the ball rests posteriorly in the chamber toward the tracheotomy tube, allowing free flow of air. As the patient inhales, incoming air flows over the ball, between the ramps, and into the patient's lungs.

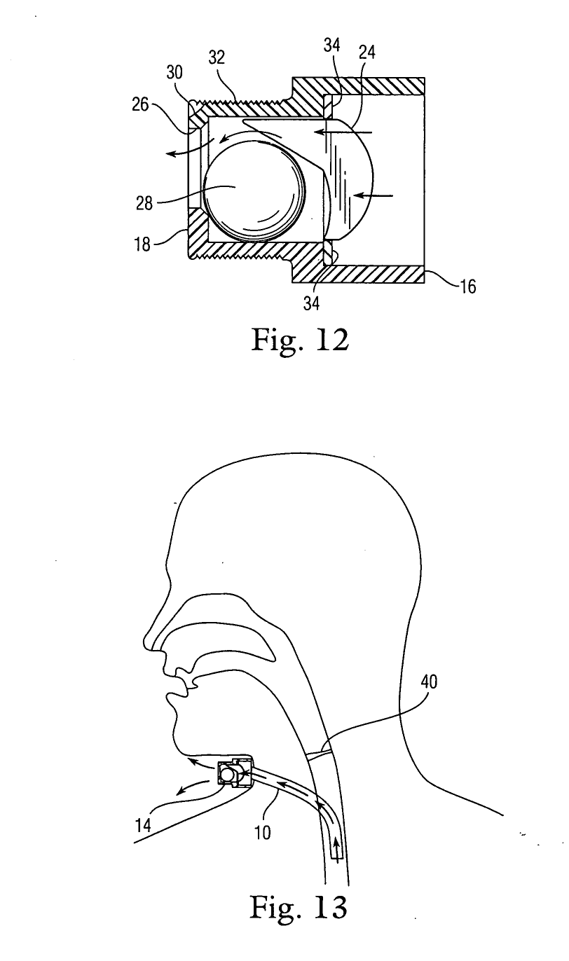

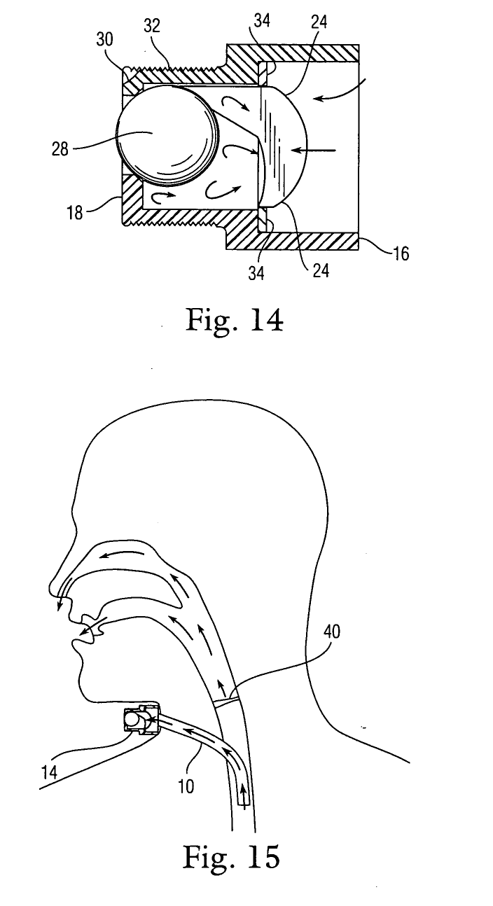

With the indexing means in the “up” position, when the patient exhales (FIGS. 12 and 13), the ball is displaced toward the frontal opening but the ball does not rise upwardly to the eccentrically formed frontal opening which is near the upper portion of the body when in the “up” position. No seal occurs in the frontal opening and air moves out of the patient's lungs, through the tracheotomy tube, and exits out of the valve.

However, with the indexing means in the “up” position, if the patient increases the exhalation force, the ball is forced upwardly in the chamber and the ball seats in the frontal hole wherein air does not flow through the valve but the air is forced through the upper airway of the patient. In this scenario, the patient can speak since air is passing over the patient's vocal cords 40.

Thus, in a preferred embodiment, the present invention allows a method of using the speaking valve in two different positions (“up” or “down”), and providing a positive ball positioning feature depending on how the housing chamber is rotated, hence greatly improving performance. In the “down” mode the ball is automatically directed forward and held fully seated towards the front opening of the valve body, when the patient is breathing regularly at rest. This innovation allows the ball to sit inside the frontal opening and provide a leak free seal to the valve with no expiratory air required to seat the ball in the opening (“biased-closed position”). In the “up” mode, the ball has a tendency to sit away from the frontal opening, closer to the posterior opening of the chamber, providing a more open airflow passage (“biased-open position”) hence allowing the patient to breathe easier. Additionally, the ball now requires a conscious effort in terms of exhalation force, to seat the ball in the frontal opening and seal off airflow. Because of this, exhaled air can either be allowed to exit through the valve rather than being redirected through the patient's upper airway. Alternately, the patient can force the ball to seal when re-direction of airflow is desired for speech production. The ramps 24 are connected to a ring 34 which is disposed against the internal step 20 such that the ramps 24 extend inwardly into the chamber in the body 14. The ring 34 is keyed and ultrasonically welded to the step of the body to retain the ramps in place and in a proper orientation (FIG. 16). The keying means 36 may be a tab formed on the ring with a cooperating notch formed in the step, or the tab may be formed on the step and the notch may be formed on the ramp's ring. Other keying means known to persons skilled in the art may be used.

In an alternate mode, the valve including the body, the ramps and the ball, is mounted in the cannula of the tracheotomy tube. Although the valve cannot be rotated, the valve operates in a manner as described above.

Having the exhaled air from the patient make contact with heat moisture exchange (HME) media is essential to the function of an HME. Because HME filters function only when air from the patient is exhaled across the media, and then returned to the patient, use of an HME is not possible with all other current unidirectional speaking valves which do not allow two-way airflow (air in and air out). Prior to the valve design of the present invention, patients had to choose either to wear a speaking valve for communication and forgo the benefit of an HME filter, or alternatively to wear an HME filter and forgo the benefits of wearing a speaking valve. In the present invention a cap 38 containing fibers for HME is removably attached to the second end of the body. The novel ball valve's guiding design is unique in a sense that when the indexing means is in the “up” position (biased-open), the ball rests posteriorly inside the chamber, greatly facilitating airflow during inhalation (FIGS. 9, 10, 11). This position accommodates the use of an HME as follows. Upon exhalation, air is uniquely allowed to flow back out through the valve, and through the HME, making contact with the filter media. In this way the patient receives the benefit of the HME filtered air upon inspiration. However, with the valve in this same position, the patient can also choose to have the ball seat and seal at will, allowing redirection of the exhaled air over the vocal cords, in order to produce speech. No repositioning of the valve itself is necessary to achieve this. This is accomplished simply by providing increased expiratory volume in order to drive the ball forward and vertically up the frontal wall, and into the frontal opening to seat the ball and seal off airflow (FIG. 15).

This speaking valve uniquely allows the tracheotimized patient to realize the benefits of both automatic speech and humidification concurrently.

Obviously, many modifications may be made without departing from the basic spirit of the present invention. Accordingly, it will be appreciated by those skilled in the art that within the scope of the appended claims, the invention may be practiced other than has been specifically described herein.

What is claimed is:

1. A speaking valve for management of a patient's airway comprising a body removably attached to a tracheotomy tube, a plurality of circumferentially-spaced ramps disposed within the body forming a chamber therein, the body having a first end communicating with the tracheotomy tube and a second end distal from the tracheotomy tube, an opening formed in the second end offset from a central axis of the chamber, a ball having a diameter disposed within the chamber eccentrically of the central axis of the chamber, the ball being guided up and down the ramps and substantially closing the opening in the second end of the body, and
means for rotating the body and hence the chamber therein, at the option and control of the patient wherein the ball moves up and down the ramps opening and closing the opening in the second end of the body and, as selected, exhalation by the patient proceeds through the patient's upper respiratory system and facilitates speaking by the patient.

2. The speaking valve of claim 1, wherein the body is rotated approximately 180° at the option of the patient.

3. The speaking valve of claim 1, wherein the means to rotate the body are formed on an outer surface of the body.

4. The speaking valve of claim 1, wherein there are two parallel ramps, the ramps being spaced apart at a distance less than the diameter of the ball to retain the ball.

5. The speaking valve of claim 4, wherein the ramps are formed having an acute angle upward toward the first end of the body.

6. The speaking valve of claim 1, wherein the body has an indexing means thereon to indicate the orientation of the body for rotation.

7. The valve of claim 1, wherein a cap containing fiber for heat moisture exchange is removably attached to the second end of the body.

8. The speaking valve of claim 1, wherein the first end of the body has first diameter and the second end of the body has a second diameter, the first diameter being larger than the second diameter, an internal step being formed within the body.

9. The speaking valve of claim 8, further comprising each ramp being connected to a ring, the ring being disposed against the internal step wherein each ramp extends inwardly into the chamber, the ring being attached to and oriented to the internal step by keying means.

10. In a speaking valve for a tracheotomy tube for a patient, the combination of a body having a chamber formed about a central longitudinal axis and having a frontal opening, a ball eccentrically disposed within the chamber and in a biased-open position, seated posteriorly inside the chamber, away from the frontal opening resulting in less interference with air flow and easier inhalation, and means for rotating the body approximately 180° about the central longitudinal axis thereof, and at the patient's or caregiver's option, to thereby automatically direct the ball forward against the frontal opening to thereby seal the frontal opening in a biased-closed position, so that the exhalation is through the patient's upper respiratory system and over the patient's vocal cords, thereby facilitating substantially-improved speech by the patient.

11. In a tracheotomy tube for a patient, the combination of a speaking valve operable in a biased-open or biased-closed position, respectively, at the option of the patient or the patient's caregiver, and a heat moisture exchange (HME) device cooperating with the speaking valve to facilitate improved airway management for the patient consonant with substantially-improved speech.

12. A speaking valve for a patient's airway management, comprising a body having a chamber with a ball disposed therein eccentrically of the central axis of the chamber, means including a plurality of circumferentially-spaced ramps for guiding the ball up the ramps upon initial inhalation by the patient, and means for rotating the body and hence the chamber therein, at the option of the patient; wherein during the exhalation process, the exhaled air causes the ball to ride up the frontal wall seating in and sealing the opening and redirecting the exhaled air to exit through the laryngeal opening, thus enabling speech.

13. The speaking valve of claim 12, wherein the body is rotated by approximately 180° and at the option of the patient.

14. The speaking valve of claim 12, wherein a cap is attached to the distal end of the body, the cap is provided with openings for air circulation, and wherein the cap contains heat moisture exchange (HME) filter material.

15. In a speaking valve for use with a tracheotomy device, the improvement comprising a body having a proximate end and a distal end, respectively, the distal end of the cylinder having an opening formed therein and the body having a ramp means formed therein, a ball disposed in the cylinder between the ramp means and the opening and trapped therein, and the ramp means including a pair of substantially-parallel ramps spaced laterally apart a distance which is less than the diameter of the ball.

16. The improvement of claim 15, further including an integrally-molded subassembly having an annular ring joined to the pair of ramps.

17. The improvement of claim 16, further including circumferential keying means between the annular ring and the cylinder.
